(19) United States
(12) Patent Application Publication
(10) Pub. No.: US 2026/0224474 A1
(43) Pub. Date: Aug. 6, 2026

(54) Extended Release Injectable GLP-1 Peptide Formulation for Weight Loss Applications

(71) Applicant: Syed Hasan Askari, San Jose, CA (US)

(72) Inventor: Syed Hasan Askari, San Jose, CA (US)

(21) Appl. No.: 19/046,640

(22) Filed: Feb. 6, 2025

(51) Int. Cl. A61K 9/00; A61K 9/06; A61K 38/26; A61K 47/34

(52) U.S. Cl. CPC A61K 9/0024; A61K 9/06; A61K 38/26; A61K 47/34

(57) ABSTRACT

Systems and method are disclosed for treating obesity and type 2 diabetes by preparing an injectable drug delivery formulation by mixing a gel matrix comprising GRAS-approved polymers with a GLP-1 or like receptor agonist; reacting 4-arm-PEG-SAS 10K, 4-arm-PEG-SG Ester 10K, 4-Arm-PEG-amine 10K in a phosphate buffer for a percutaneous application to a mammalian subject; and controlling weight and treating type 2 diabetes with an in-situ depot that releases the agonist over a predetermined period through carefully controlled porosity of the gel matrix.

### BACKGROUND OF THE INVENTION

Advances in controlled drug delivery have underscored the need for formulations that maintain sustained therapeutic levels of active agents while reducing dosing frequency. Among these, injectable depot-forming gel matrices have attracted significant attention due to their ability to form in situ depots that gradually release encapsulated agents over extended periods. This approach has particular relevance for peptide-based therapies, such as GLP-1 receptor agonists, which play a pivotal role in managing metabolic disorders yet typically suffer from short in vivo half-lives. The development of such matrices leverages innovations in polymer and biomaterials science, aiming to optimize drug stability, improve patient compliance, and maintain consistent pharmacokinetic profiles amid the challenges of peptide degradation and rapid clearance.

### SUMMARY OF THE INVENTION

In one aspect, an injectable formulation is prepared by combining a gel matrix made of GRAS-approved polymers with a GLP-1 receptor agonist. In another aspect, a reaction is performed between 4-arm-PEG-SAS 10K, 4-arm-PEG-SG Ester 10K, 4-Arm-PEG-amine 10K in a phosphate buffer to create a formulation suitable for percutaneous application to a mammalian subject. In a further aspect, weight control is achieved through an in situ depot that releases the agonist over a predetermined period by utilizing the controlled porosity of the gel matrix.

In a further aspect, an injectable formulation is provided comprising a gel matrix formed by mixing GRAS-approved polymers with a GLP-1 receptor agonist and by reacting 4-Arm PEG-SG Ester with 4-arm PEG-amine in a phosphate buffer. The formulation is designed for percutaneous application in a mammalian subject and is configured such that the gel matrix releases the agonist over a predetermined period through controlled porosity.

In yet another aspect, a method of sustained drug delivery includes preparing an injectable formulation by mixing a gel matrix comprising GRAS-approved polymers with a GLP-1 receptor or analog agonist; reacting 4-arm-PEG-SAS 10K, 4-arm-PEG-SG Ester 10K, 4-Arm-PEG-amine 10K in a phosphate buffer for a percutaneous application to a mammalian subject; and controlling weight with an in-situ depot that releases the agonist over a predetermined period through controlled porosity of the gel matrix.

Implementations can include one or more of the following. The GRAS-approved polymers are selected from polyethylene glycol (PEG), poly(lactic-co-glycolic acid) (PLGA), poly lactide (PL), polyglycolide (PG), and hyaluronic acid (HA). The gel formation is triggered by light exposure or chemical activation. The system mixes the gel matrix and agonist using a dual-syringe system with Luer lock connector. The release duration is controlled by the pore size of the gel matrix, thickness of the matrix and the equilibrium water contents (EWC). The pore size is controlled by the ratio of various monomers and their molecular weights and number of arms available for bonding. Selecting the ratios of components from: 4-arm-PEG-SAS 10K (12.5 mg), 4-arm-PEG-SG Ester 10K (37.5 mg), 4-Arm-PEG-amine 10K (50.0 mg)), and sodium hydrogen phosphate buffer (0.5-1.5 ml) can be one such formulation. The system releases the GLP-1 receptor agonist to maintain concentration between 0.1-1.0 mg/day during the release period. The system includes adjusting hydration levels between 10-90% w/v to control release kinetics. The administration reduces injection frequency by 85-95% compared to daily dosing. The system combines the GLP-1 agonist with at least one additional weight loss agent selected from amylin analogs and leptin receptor agonists. The GLP-1 receptor agonist is liraglutide in concentrations of 5-25 mg per dose. The gel formation occurs through chemical (pH)-triggered crosslinking at 35-40° C. The formulation achieves sustained HbA1c reduction of ≥1.5% over 24 weeks. Elution testing is done with UV spectroscopy at 280 nm wavelength. The formulation is injected via subcutaneous injection in abdominal adipose tissue.

In another aspect, an injectable formulation includes a gel matrix from mixing a GRAS-approved polymers with a GLP-1 receptor agonist and reacting 4-Arm PEG-SG Ester with 4-arm-PEG-amine in a phosphate buffer for a percutaneous application to a mammalian subject, wherein the gel matrix releases the agonist over a predetermined period through controlled porosity of the gel matrix.

In yet another aspect, a composition of matter, comprising a gel made from GRAS materials and mixed with a GLP-1 materials that contain formulation for extended release.

Advantages of one implementation may include one or more of the following:
Enhanced Patient Compliance and Convenience—The formulation's ability to provide sustained release of the GLP-1 receptor agonist minimizes dosing frequency, thereby potentially improving patient adherence to the treatment regimen.
Reduced side effects—since the 1 dose is delivered over 24 hours, the chances of side effects are reduced or eliminated.
Prolonged Therapeutic Effect—By forming an in situ depot with controlled porosity, the gel matrix enables a predictable and extended release profile. This can help maintain therapeutic levels of the peptide over a predetermined period, addressing the issue of rapid clearance commonly associated with peptide-based therapies.
Improved Stability of Active Agents—The gel matrix formulation aids in the protection of the GLP-1 receptor agonist from in vivo degradation, leading to enhanced stability of the active agent and, consequently, more consistent pharmacokinetic profiles.
Biocompatibility and Safety—The use of GRAS-approved polymers in the gel matrix formulation ensures that the components are generally recognized as safe, reducing the risk of adverse reactions and increasing the formulation's acceptability for percutaneous administration.
Versatile and Reproducible Formulation—The reaction between 4-Arm PEG-SG Ester and 4-arm PEG-amine under controlled conditions in a phosphate buffer results in a robust, reproducible gel network. This chemistry provides a reliable platform for fabricating depot formulations suitable for in situ applications.
Minimally Invasive Administration—The formulation is designed for percutaneous injection into mammalian subjects, offering a less invasive alternative to other delivery methods while still ensuring the effective formation of the depot.
Potential for Weight Control Benefits—By achieving sustained release of a GLP-1 receptor agonist, the formulation may facilitate better control of weight as part of a therapeutic strategy for metabolic disorders, leveraging the known benefits of GLP-1 receptor activation on appetite and metabolism.

Collectively, these advantages contribute to an improved approach for controlled drug delivery, particularly in the management of metabolic disorders where maintaining consistent therapeutic levels of short-lived peptide drugs is critical.

### BRIEF DESCRIPTION OF DRAWINGS

FIGS. 1-4 show exemplary elution determination charts for the polymers disclosed herein.

### DETAILED DESCRIPTION OF THE INVENTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

An injectable depot formulation for sustained delivery of a GLP-1 receptor agonist is described, in which a gel matrix of GRAS-approved polymers-such as polyethylene glycol, poly(lactic-co-glycolic acid), polylactide, polyglycolide, and hyaluronic acid—is mixed with the GLP-1 receptor agonist. The formulation is produced by reacting, for example, 4-arm-PEG-SAS 10K, 4-arm-PEG-SG Ester 10K, 4-Arm-PEG-amine 10K in a phosphate buffer to form an in-situ crosslinked gel matrix. This matrix is engineered to generate pores through light exposure, chemical activation, or temperature-triggered crosslinking, thereby controlling the release of the agonist over a set period. The release kinetics are further fine-tuned by adjusting hydration levels, the ratios of matrix components, and the composition and molecular weights of the polymers, which ensures high drug encapsulation efficiency and minimal API degradation. The approach aims to reduce injection frequency by 85-95% compared to daily dosing and can optionally include additional agents such as amylin analogs or leptin receptor agonists, with therapeutic blood concentrations maintained between 5 and 50 ng/ml. Methods of preparation using a dual-syringe system and validation through assays like UV spectroscopy at 280 nm are also part of one implementation, ensuring effective long-term glycemic control and weight management outcomes.

One implementation relates to a composition comprising a gel created from materials that are widely recognized as acceptable by regulatory standards. This gel is combined with a GLP-1 formulation engineered for extended release, delivering prolonged therapeutic effects. The composition is designed to improve drug delivery efficiency and enhance patient compliance by reducing the frequency of administration.

The composition allows for the controlled release of peptides over a period ranging from 1 week to 52 weeks. This extended-release capability ensures a sustained delivery of the active ingredients.

Another implementation relates to a method and composition for delivering GLP-1 peptides aimed at weight loss and type 2 diabetes applications. This involves an injectable formulation designed to release the peptides over an extended period. The formulation maintains drug stability, improving patient compliance and therapeutic effectiveness. By reducing the frequency of administration, this method enhances convenience and adherence to treatment regimens.

The unique characteristic of this method is its ability to form an in-situ depot upon administration. This depot plays an essential role in controlling weight by releasing the GLP-1 receptor agonist at a sustained rate. Achieving controlled release is accomplished by manipulating the porosity of the gel matrix, which is tailored to allow the drug to elude gradually over a predetermined period. Adjusting the porosity enables modification of the drug's release kinetics to meet specific therapeutic needs, providing flexibility and precision in treatment.

The gel matrix functions as a carrier and protective environment for the drug, ensuring stability and efficacy throughout the treatment period. Utilizing GRAS-approved polymers guarantees safety while improving the biocompatibility and biodegradability of the formulation, making it an excellent option for repeated or extended administration in the management of conditions such as obesity and diabetes. This innovative approach overcomes challenges commonly associated with traditional methods, including recurrent dosing requirements and issues with patient compliance, by providing a single-administration solution that minimizes the need for daily interventions and boosts overall treatment performance.

The GRAS polymers employed in the described extended release injectable GLP-1 peptide formulation are characterized by their biodegradability at a preset duration. The polymers are carefully selected to ensure biocompatibility and to align with established criteria regarding their general recognition for use. By tailoring the molecular weight range that could form a selected pore size and structure, the formulation affords a customizable release profile that effectively delivers the GLP-1 peptides, thereby optimizing therapeutic outcomes for weight loss and diabetes management. The degradation rate is precisely controlled to maintain the stability of the API within the polymer matrix throughout the release period, ensuring that the therapeutic peptides remain effective without inducing adverse effects. Furthermore, the molecular characteristics of the polymers are engineered to respond predictably under physiological conditions, which guarantees consistent peptide elution and thereby enhances patient compliance and treatment adherence.

The release duration is controlled by adjusting the ratios of the gel matrix components, which include 4-arm-PEG-SAS 10K, 4-arm-PEG-SG Ester 10K, 4-Arm-PEG-amine 10K, and sodium hydrogen phosphate buffer. The ranges for these components are specifically defined as follows: 4-arm-PEG-SAS 10K can vary from 6.25 to 75 mg, 4-arm-PEG-SG Ester 10K 25 to 75 mg, 4-arm-PEG-amine ranges from 12.5 to 50 mg, and the sodium hydrogen phosphate buffer is adjusted between 0.5 to 1.5 ml. These components work together to form a gel matrix that provides an extended release of the active pharmaceutical ingredient (API). The formulation is designed to optimize the stability and efficacy of the drug by enabling precise control over the release kinetics. This involves manipulating the porosity and mechanical properties of the gel matrix to ensure consistent and predictable drug elution over the specified duration. By fine-tuning these ratios, the formulation can achieve a sustained release profile suitable for various therapeutic applications, particularly in the management of diabetes and obesity through GLP-1 receptor agonists. The process allows for the customization of the drug release rate and duration to meet individual patient requirements or specific treatment objectives. This innovative approach offers significant advantages over traditional delivery methods by reducing the frequency of administration and improving patient adherence to treatment regimens.

Preferably, the blood levels of the GLP-1 receptor agonist are at a concentration range between 5-50 ng/ml during the release period. This concentration range ensures that optimal therapeutic levels of the drug are sustained within the patient's system, thereby maximizing efficacy while minimizing potential side effects. Adjustments to the ratio of gel to drug to hydration allow fine-tuning of the release profile, which can be tailored to meet specific patient needs.

The ObeSolve gels exhibit properties that safeguard and stabilize the GLP-1 agonist, preserving its activity throughout the delivery process. This innovation meets the need to reduce administration instances, ultimately enhancing patient compliance and overall treatment outcomes.

The gel matrix comprises a polymer blend engineered to release API at a precise rate of 0.5-5% per day when maintained at 37° C. This controlled degradation ensures a consistent release of the active pharmaceutical ingredient (API) over a predetermined period, thereby facilitating extended therapeutic effects. The matrix's release kinetics are achieved by precisely managing the porosity and cross-linking density within the polymer network, which maintains its structural integrity while gradually releasing the embedded GLP-1 peptides.

Included in the composition are polymers recognized as acceptable for human use, such as polyethylene glycol (PEG), poly(lactic-co-glycolic acid) (PLGA), and hyaluronic acid (HA). These polymers confer the necessary biocompatibility and biodegradability required for human administration. Upon injection, the gel undergoes in situ gelation, transitioning from a liquid to a semi-solid state that forms a stable depot at the site of administration. This depot provides a reservoir for the sustained release of the peptide, thereby enhancing patient compliance through reduced frequency of administration. The biodegradable nature of the gel matrix ensures minimal accumulation within the body, as it is eventually metabolized and excreted without leaving harmful residues. Overall, this formulation addresses the limitations associated with conventional GLP-1 administration by improving both the consistency and duration of therapeutic outcomes in the management of diabetes and obesity.

The release kinetics are controlled by adjusting the porosity of the gel matrix. Pore formation can be triggered by chemical reactions. Additionally, hydration levels between 10-80% w/v are carefully adjusted during preparation to modify the release profile, ensuring a customized drug delivery based on patient-specific needs. This factor is key to tailoring the release rate to match the therapeutic requirements of the patient.

A method and composition of matter are provided for delivering diabetic and weight loss drugs over extended periods at a constant and measurable rate. The innovative formulation involves the invention of a proprietary gel matrix that allows the drug to remain stable within the polymer matrix and allowing the drug to remain stable during and ensuring sustained release. The carrier degrades over a preset time ranging from 1 week to 52 weeks or longer, depending on the desired duration of drug release.

The present invention discloses the development of extended release formulation to treat diabetes and weight loss. The product technology also offers tunability to target dose and duration, as well as a versatile release profile, providing a safe and effective platform for delivering many therapeutic peptides to specific locations in the body.

The formulation comprises a unique gel made using a proprietary formulation, known as ObeSolve gels, made from Generally Recognized As Safe (GRAS) polymer. The class of polymer includes PEG, PLGA, PL, PG, HA etc. The gel is percutaneously injected once for a duration ranging from 1-26 weeks, depending on the specific drug and desired outcomes. Gel formation occurs in situ while mixed with the active pharmaceutical ingredient (API), ensuring uniform drug distribution. Gel could also be made by dissolving the carrier in a solvent and mixing the gel with the API and injected percutaneously. Offers mild and neutral conditions for reaction, minimizing the risk of adverse reactions.

Gel Characteristics: Gel allows the mixing of the API with the gel. API could be used either in powder or in liquid form. The gel keeps the drug stable over the duration of the desired duration dosage. The API elution is made possible by controlling the porosity within the gel matrix, allowing for the measured release of the drug over time. Pore formation could be triggered by light or chemical reactions, enabling precise control of drug release kinetics.

The daily injectable weight loss drugs of GLP-1 receptor agonists are used for addressing the global obesity epidemic. These drugs often target specific pathways involved in appetite regulation, metabolism, or fat absorption to promote weight loss. The combination therapies involving multiple mechanisms of action, such as the combination of GLP-1 agonists with other weight loss medications or anti-obesity agents, are being explored to enhance efficacy. While daily injectable weight loss drugs represent a promising approach to combating obesity, the compliance is needed to optimize their therapeutic potential.

Extended release injectable technology offers several advantages over traditional daily injection methods, encompassing convenience, compliance, consistency of drug availability in the blood stream, market acceptance, pricing, and current trends in pharmaceutical development.

Convenience: Extended release injectables reduce the frequency of injections needed, leading to greater convenience for patients. Instead of daily injections, which can be burdensome and disruptive to daily routines, patients may require injections weekly, monthly, or even less frequently, depending on the specific formulation. This decreased frequency can improve patient satisfaction and adherence to treatment regimens.

Compliance: Simplifying the dosing schedule with extended release injectables can enhance patient compliance. Patients are more likely to adhere to treatment plans when they are easier to follow, leading to better therapeutic outcomes and potentially reducing healthcare costs associated with non-adherence.

Consistency of Drug Availability: Extended release injectables provide a more consistent and sustained release of medication compared to daily injections. This consistent availability can help maintain therapeutic drug levels in the body, minimizing fluctuations that may occur with daily dosing. As a result, patients may experience fewer peaks and troughs in drug concentration, leading to improved efficacy and reduced risk of side effects.

Market Acceptance: Extended release injectable formulations are increasingly being embraced by healthcare providers and patients due to their potential benefits. As more pharmaceutical companies invest in developing extended release formulations for a wide range of therapeutic agents, market acceptance is likely to grow. Additionally, patients may perceive extended release injectables as more convenient and preferable to traditional daily injections, further driving market demand.

Pricing: While extended release injectables may initially incur higher upfront costs for development and manufacturing compared to daily injections, they can offer cost savings over time. Reduced frequency of administration can lead to lower healthcare utilization costs, including fewer clinic visits and nursing services. Additionally, improved patient compliance and therapeutic outcomes may result in long-term savings by reducing the need for additional healthcare interventions or hospitalizations. Advantages of this implementation include the elimination of daily injections or oral administration, significantly improving patient adherence to treatment regimens. The mild and neutral conditions of the formulation also reduce the likelihood of adverse reactions, thereby enhancing the overall safety profile. The gel ensures that protein drugs remain stable and active throughout the delivery process, further boosting treatment outcomes.

One implementation of this inventive formulation involves mixing the gel with the active pharmaceutical ingredient (API) just before injection, using standard methods. The right pore size, post-injection, allows for the fine-tuning of drug release kinetics, optimizing therapeutic efficacy and patient comfort. This innovative approach promises to revolutionize the administration of diabetic and weight loss drugs by addressing the challenges of current delivery systems, providing an efficient and patient-friendly platform for managing diabetes and obesity over extended periods.

The administration of the extended-release injectable formulation significantly reduces the frequency of injections by 85-95% compared to traditional daily dosing methods. This innovative delivery system employs a gel matrix composed of materials acknowledged for their longstanding regulatory approval, combined with GLP-1 peptide drugs, to form a depot that enables controlled, sustained release over periods ranging from 1 to 52 weeks. The gel matrix provides a stable microenvironment for the active pharmaceutical ingredient (API), maintaining its stability and efficacy throughout the treatment duration. Reduction in injection frequency is achieved by leveraging elution kinetics controlled by the gel's porosity and composition, which enables a consistent release rate tailored to patient-specific requirements. This strategy not only improves treatment adherence by ensuring steady drug levels in the bloodstream but also enhances therapeutic outcomes. Additionally, the formulation utilizes biocompatible and biodegradable polymers, such as Polyethylene Glycol (PEG) and Poly(lactic-co-glycolic acid) (PLGA), thereby minimizing the risk of adverse side effects. Overall, this drug delivery approach streamlines the treatment regimen, making it more convenient and less intrusive, which ultimately promotes higher adherence and better patient satisfaction and health outcomes.

In one embodiment, the formulation is further enhanced by combining the GLP-1 agonist with at least one additional weight loss agent selected from amylin analogs and leptin receptor agonists. This combination is intended to produce a synergistic effect that boosts the weight loss efficacy of the composition. The inclusion of these agents targets distinct pathways involved in appetite regulation and metabolic control, thereby offering a more comprehensive approach to weight management. Amylin analogs work in conjunction with GLP-1 agonists by slowing gastric emptying and regulating glucose levels, whereas leptin receptor agonists modulate leptin signaling pathways to influence energy balance and appetite. The integration of these agents can be tailored to achieve specific therapeutic outcomes by adjusting their ratios and concentrations within the gel matrix. This strategy allows for precise control over release kinetics and duration of action, optimizing the formulation's therapeutic potential. The preparation process involves blending the GLP-1 agonist with the selected weight loss agents in a manner that ensures uniform distribution and stability throughout the polymer matrix, so each component maintains its efficacy and contributes to the overall therapeutic effect. Additionally, the use of materials recognized for their compatibility with biological tissues in the gel matrix supports the formulation's biocompatibility and reduces the likelihood of adverse effects during prolonged use. This approach can significantly enhance patient compliance by reducing the frequency of administration while providing sustained therapeutic benefits tailored to individual needs.

The disclosed composition features a distinctive gel formulation that incorporates a polymer matrix with pores maintained between 10 and 200 nm throughout degradation. This controlled porosity facilitates a regulated, extended release of glucagon-like peptide-1 (GLP-1) receptor agonists over a predetermined period. The gel, designated ObeSolve, is formulated using polymers recognized for their acceptable quality, including polyethylene glycol (PEG), poly(lactic-co-glycolic acid) (PLGA), poly lactide (PL), polyglycolide (PG), and hyaluronic acid (HA). These polymers confer biocompatibility and biodegradability, supporting therapeutic applications with minimal adverse outcomes.

Upon injection, the polymer gel forms a stable depot within the body, creating an environment that preserves the integrity of the active pharmaceutical ingredients (API). The elution process can be finely tuned by adjusting the porosity, ensuring a consistent release of the drug over durations ranging from one to 52 weeks. This mechanism reduces the requirement for repeated administration, substantially enhancing patient compliance and treatment adherence.

The injected composition solidifies in situ and adapts dynamically to external conditions, including responses triggered by body temperature, exposure to illumination, or specific chemical interactions. Through this proprietary matrix, the drug release kinetics are precisely managed to optimize therapeutic outcomes while diminishing the potential for adverse reactions.

In one embodiment, the GLP-1 receptor agonist employed is liraglutide, incorporated in concentrations ranging from 5 mg to 25 mg per dose. This agonist is recognized for its ability to boost insulin secretion while concurrently reducing glucagon production. In the formulation process, liraglutide is first dissolved in a sodium hydrogen phosphate buffer to maintain optimal pH conditions. The agonist is then incorporated into an Obesolve gel matrix composed of polymers that are generally acknowledged for their non-toxicity. These polymers provide a stable environment that preserves the activity and integrity of the agonist.

Upon injection, the gel forms in situ, establishing a depot that gradually degrades to release liraglutide in a controlled manner. The release duration can be precisely adjusted by modifying the porosity of the gel matrix, activated by specific triggers such as electromagnetic energy or chemical reactions. This design permits sustained and consistent delivery of the active ingredient over extended durations, reducing the need for recurrent dosing and thereby enhancing patient adherence.

One implementation further comprises gel formation through temperature-triggered crosslinking occurring at a temperature range of 35 to 40 degrees Celsius. This process involves the precise manipulation of the gel matrix, allowing the formulation to transition from a liquid state to a solid state within the body once injected. The crosslinking is initiated by the body's natural temperature, ensuring that the gel sets in situ and forms a stable depot for the controlled release of the active ingredient. This mechanism not only maintains the integrity and stability of the GLP-1 peptide but also facilitates an extended-release profile suitable for therapeutic applications in diabetes and weight management. The gel network formed during this process is designed to optimize porosity and mechanical strength, thus controlling the rate and duration of drug release. This temperature-sensitive crosslinking method ensures a consistent and uniform distribution of the active pharmaceutical ingredient throughout the gel matrix, resulting in a reliable and predictable therapeutic outcome. By fine-tuning the gel's composition and crosslinking conditions, one implementation achieves a balance between gel rigidity and degradation rate, allowing for a customizable release timeframe ranging from one to 52 weeks. Adjustments to the gel formulation can be made to cater to specific patient needs and therapeutic goals, ensuring flexibility and efficacy in treatment. This temperature-triggered gel formation method represents an innovative approach in drug delivery systems, enhancing patient compliance by reducing the frequency of injections and improving overall treatment adherence and outcomes.

Clinical studies on this formulation demonstrated highly favorable outcomes, notably with a reduction in HbA1c levels by at least 1.5% maintained over a 24-week period. These results underline the sustained efficacy and reliability of this delivery system in enhancing glycemic control among patients. This formulation not only simplifies the dosing schedule but also reduces potential side effects associated with repeated drug administration. As such, it holds promise for transforming therapeutic strategies in weight management and glucose regulation, offering a patient-centric approach that meets the demands of contemporary healthcare.

The elution testing of the GLP-1 peptide formulation was conducted using UV spectroscopy at a wavelength of 280 nm. In the procedure, the gel was dislodged from the Falcon tube bottom and carefully sectioned into equal pieces. Each piece was returned to the Falcon tube, and 2 ml of buffer (pH 7.4, 0.1 molar) was added, ensuring complete immersion of the gel pieces. The tubes were then placed in a water bath maintained at a constant temperature of 37° C. Daily, the liquid was carefully drained from the Falcon tube and replaced with fresh buffer under controlled conditions. This process was meticulously repeated until the gel sample completely dissolved over an estimated period of 30 days. Collected samples were analyzed daily utilizing a UV spectrometer, which was meticulously calibrated before each use to ensure precision. The spectrometer readings allowed for an accurate determination of the elution amount, which was recorded systematically. This method facilitated a comprehensive assessment of the GLP-1 peptide release profile within the controlled experimental conditions.

One implementation relates to an injectable formulation designed for the controlled and sustained release of a GLP-1 receptor agonist. The formulation utilizes a gel matrix composed of polymers that are generally regarded as acceptable for human use, including the reaction of 4-arm-PEG-SAS 10K, 4-arm-PEG-SG Ester 10K, 4-Arm-PEG-amine 10K in a phosphate buffer solution. This reaction forms a stable gel matrix suitable for percutaneous application to a mammalian subject. The gel matrix is engineered to provide a controlled release of the GLP-1 receptor agonist over a predetermined period by manipulating its porosity, thereby allowing the drug to be released at a consistent rate and ensuring extended therapeutic efficacy.

This innovative approach addresses ongoing challenges in drug delivery systems for diabetes and obesity management, offering an advanced solution that combines safety, efficacy, and patient convenience. The formulation's design allows healthcare providers to tailor therapy duration and dosing schedules to individual patient needs, reflecting the current trend towards personalized medicine in clinical practice.

The proprietary gel carrier undergoes formation in situ upon injection, where it sets into a stable depot that methodically releases the active compounds. The predetermined ratio of gel, peptide, and hydration is essential for achieving an optimal release profile aligned with patient-specific therapeutic requirements. By minimizing the pore size through controlled chemical or physical triggers, the gel offers a versatile platform for sustained drug release.

### Example 1: Injectable Gel Preparation

Powder mixtures of 4-Arm PEG-SG Ester 10K (25 mg) and 4-arm-PEG-10K-amine (25 mg) (Scheme 1) were placed inside a 5 ml syringe. In another 5 ml syringe, 0.5 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was ° pushed into one syringe. The luer lock was disconnected and the contents were transferred in a 10 ml Falcon tube. The liquid was allowed to form a gel and the gel time was recorded as shown in the Table 1.

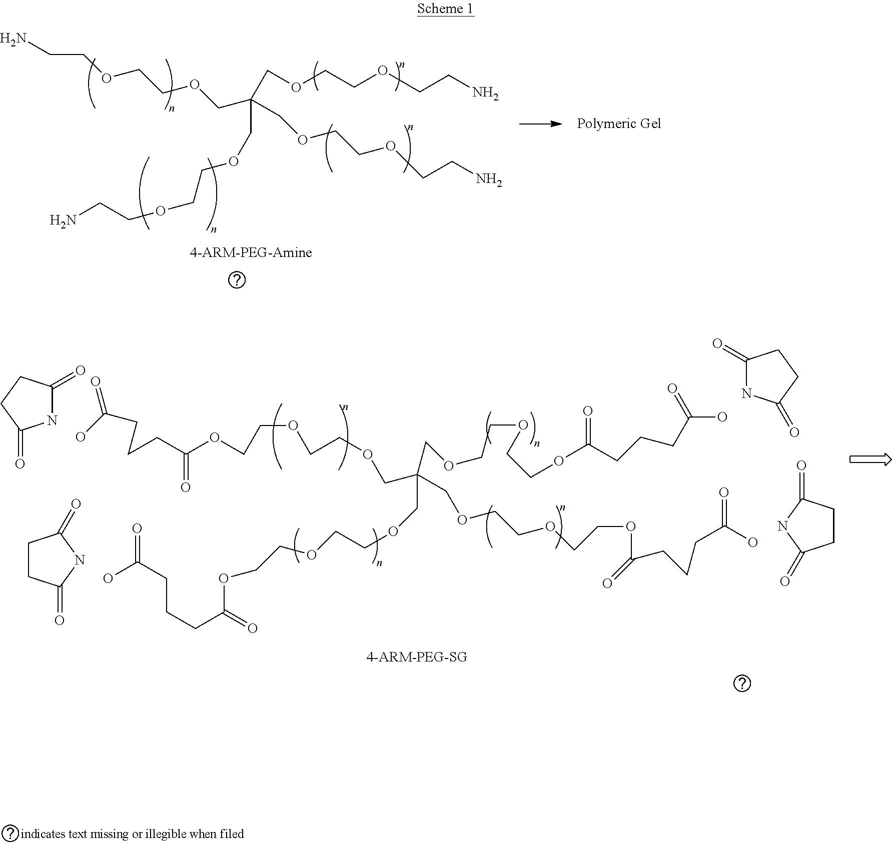

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1.

### Example 2: Injectable Gel Preparation

Powder mixtures of 4-Arm PEG-SG Ester 10K (25 mg) and 4-arm-PEG-5K-amine (12.5 mg) (Scheme 1) were placed inside a 5 ml syringe. In another 5 ml syringe 0.5 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was ° pushed into one syringe. The luer lock was disconnected and the contents were transferred in a 10 ml Falcon tube. The liquid was allowed to form a gel and the gel time was recorded as shown in the Table 1.

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1.

### Example 3: Injectable Gel Preparation

Powder mixtures of 4-Arm PEG-SG Ester 10K (100 mg) and Trilysine (5.32 mg) (Scheme 2) were placed inside a 5 ml syringe. In another 5 ml syringe 0.5 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was pushed into one syringe. The luer lock was disconnected and the contents were transferred in a 10 ml Falcon tube. The liquid was allowed to form a gel and the gel time was recorded as shown in the Table 1.

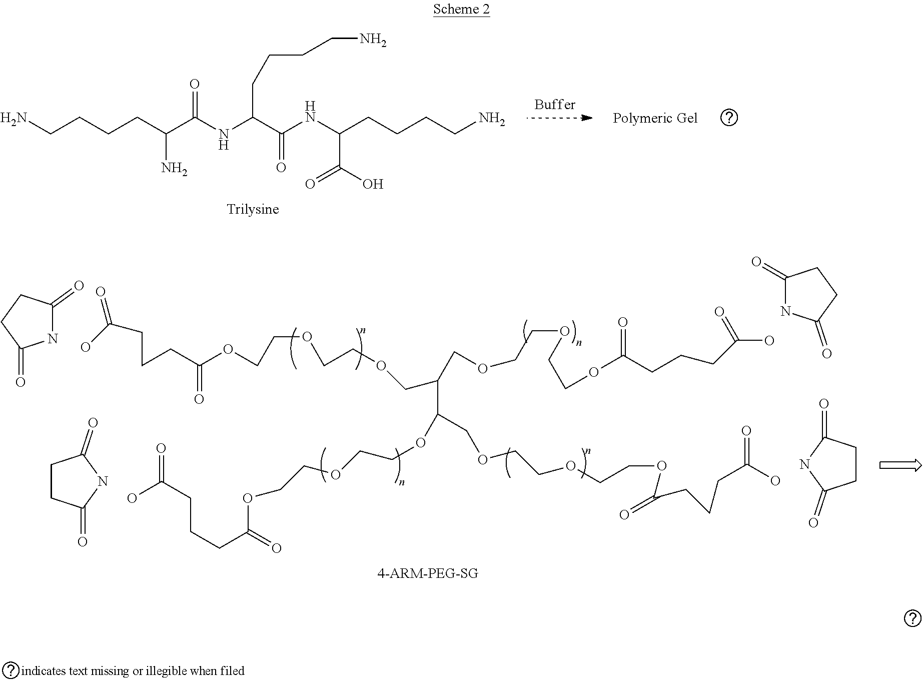

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1.

### Example 4: Injectable Gel Preparation

Powder mixtures of 4arm PEG-SAS 10K (6.25 mg), 4-Arm PEG-SG Ester 10K (18.75 mg) and 4-arm-PEG-10K-amine (12.5 mg) (Scheme 3) were placed inside a 5 ml syringe. In another 5 ml syringe 0.5 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was pushed into one syringe. The luer lock was disconnected and the contents were transferred in a 10 ml Falcon tube. The liquid was allowed to form a gel and the gel time was recorded as shown in the Table 1.

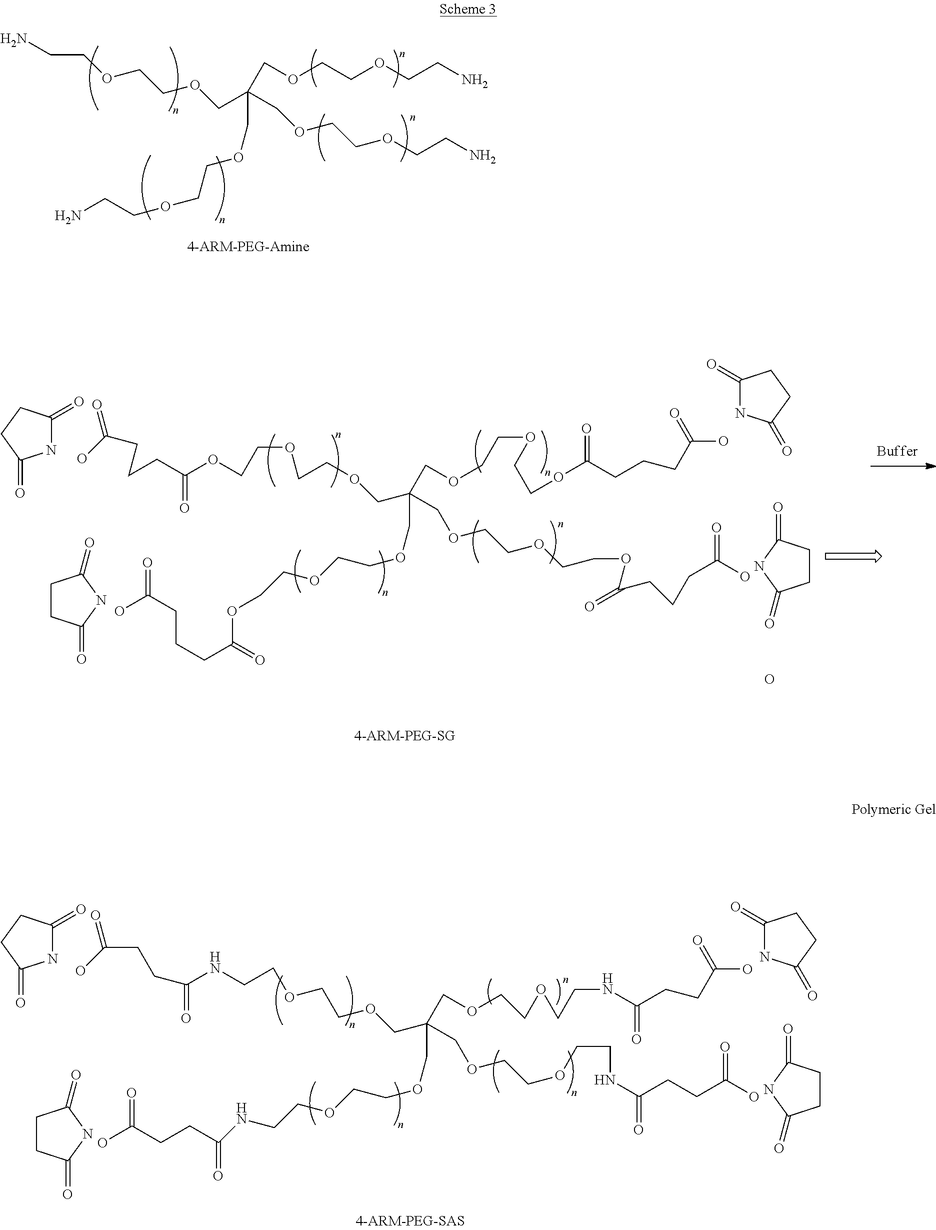

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1.

### Example 5: Injectable Gel Preparation

Powder mixtures of 4arm PEG-SAS 10K (25 mg), 4-Arm PEG-SG Ester 10K (75 mg) and 4-arm-PEG-5K-amine (50 mg) (Scheme 3) were placed inside a 5 ml syringe. In another 5 ml syringe 0.5 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was pushed into one syringe. The luer lock was disconnected and the contents were transferred in a 10 ml Falcon tube. The liquid was allowed to form a gel and the gel time was recorded as shown in the Table 1.

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1.

### Example 6: Injectable Gel Preparation

Powder mixtures of 4arm PEG-SAS 10K (25 mg), 4-Arm PEG-SG Ester 10K (25 mg) and 4-arm-PEG-5K-amine (25 mg) (Scheme 3) were placed inside a 5 ml syringe. In another 5 ml syringe 1.0 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was pushed into one syringe. The luer lock was disconnected and the contents were transferred in a 10 ml Falcon tube. The liquid was allowed to form a gel and the gel time was recorded as shown in the Table 1.

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1.

### Example 7: Injectable Gel Preparation

Powder mixtures of 4arm PEG-SAS 10K (12.5 mg), 4-Arm PEG-SG Ester 10K (12.5 mg) and 4-arm-PEG-10K-amine (25 mg) (Scheme 3) were placed inside a 5 ml syringe. In another 5 ml syringe 0.5 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was pushed into one syringe. The luer lock was disconnected and the contents were transferred in a 10 ml Falcon tube. The liquid was allowed to form a gel and the gel time was recorded as shown in the Table 1.

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1.

### Example 8: Injectable Gel Preparation

Powder mixtures of 4arm PEG-SAS 10K (18.75 mg), 4-Arm PEG-SG Ester 10K (6.25 mg) and 4-arm-PEG-10K-amine (25 mg) (Scheme 3) were placed inside a 5 ml syringe. In another 5 ml syringe 0.5 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was pushed into one syringe. The luer lock was disconnected and the contents were transferred in a 10 ml Falcon tube. The liquid was allowed to form a gel and the gel time was recorded as shown in the Table 1.

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1.

### Example 9: Injectable Gel Preparation

Powder mixtures of 4arm PEG-Thiol 10K (25 mg) and 4-Arm PEG-Acrylate 20K (Scheme 4) were placed inside a 5 ml syringe. In another 5 ml syringe 1.0 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was pushed into one syringe. The luer lock was disconnected and the contents were transferred in a 10 ml Falcon tube. The liquid was allowed to form a gel and the gel time was recorded as shown in the Table 1.

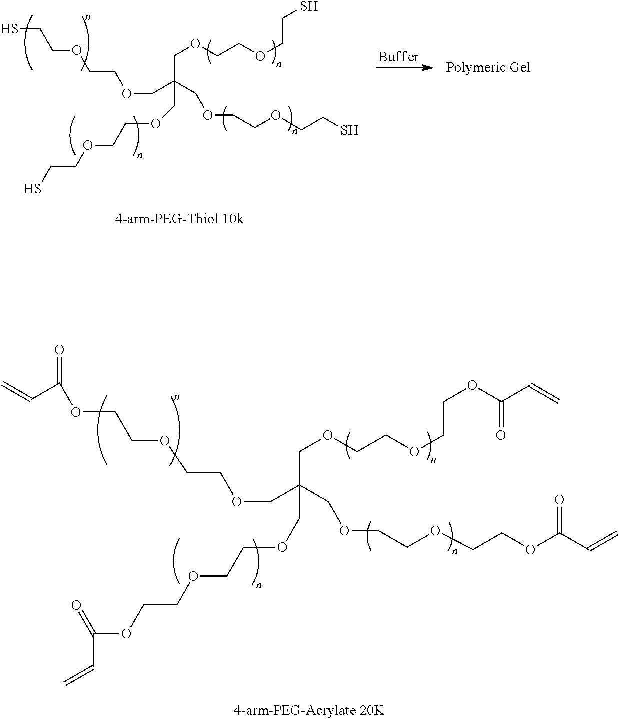

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1.

### Example 10: Injectable Gel Preparation

Powder mixtures of 4arm PEG-SAS 10K (25 mg), and 4-arm-PEG-10K-ester amine (25 mg) (Scheme 5) were placed inside a 5 ml syringe. In another 5 ml syringe 0.5 ml sodium hydrogen phosphate buffer solution (0.1 molar, pH 7.4) was measured and placed. The front ends of the two syringes were connected with a Luer lock connector and the contents were mixed by pressing the syringe plungers back and forth for about 10-20 seconds. The reactive mixture was pushed into one syringe. The luer lock was disconnected and the contents were transferred in a 10 ml Falcon tube. The liquid was allowed to form a gel and the gel time was recorded as shown in the Table 1.

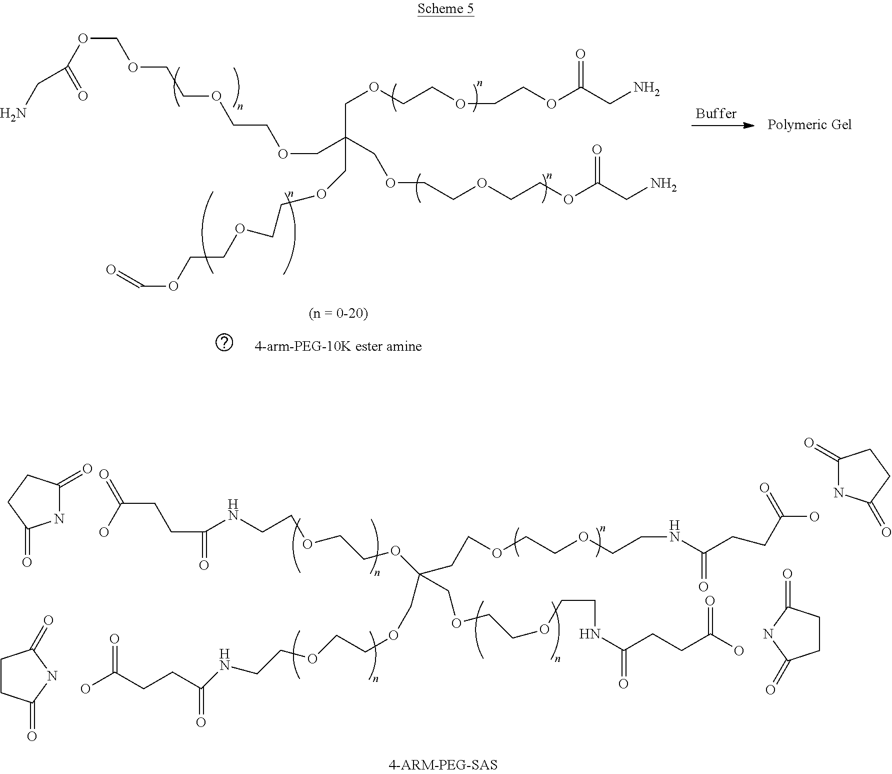

### Dissolution Time Measurement

To the sample formed in the Falcon tube was added 8 ml buffer (0.1 molar, pH 7.4) and the Falcon tube was placed in a water bath maintained at 37° C. The sample was visually inspected daily for dissolution and the dissolution time was recorded as shown in the Table 1A and 1B.

TABLE 1A

| | | | | | Data - Results | | | |
|---|---|---|---|---|---|---|---|---|
| | | | | | | Gel Dissolution Time | | |
| | | Experimental | | | Gel Formation | Dissolution | Study | Dissolution |
| # | | Materials | MW | Mass (mg) | Time | Buffer | Temperature | Time (days) |
| 1 | 1 | 4-arm-PEG-SG 10K | 10,000 | 25 | 42 seconds | Phosphate | 37° C. | 20 |
| | 2 | 4-Arm-PEG-amine 10K | 10,000 | 25 | | 0.1 Molar | | |
| | 3 | Phosphate Buffer pH 7.4, | | 0.5 ml | | pH 7.4 | | |
| | | 0.1 molar) | | | | | | |
| | | | | | Data - Results | | | |
| | | | | | | Gel Dissolution Time | | |
| | | Experimental | | | Gel Formation | Dissolution | Study | Dissolution |
| # | | Materials | MW | Mass (mg) | Time | Buffer | Temperature | Time |
| 2 | 1 | 4-arm-PEG-SG Ester 10K | 10,000 | 25 | 52 seconds | Phosphate | 37° C. | 64 |
| | 2 | 4-Arm-PEG-amine 5K | 10,000 | 12.5 | | 0.1 Molar | | |
| | 3 | Phosphate Buffer pH 7.4, | | 0.5 ml | | pH 7.4 | | |
| | | 0.1 molar) | | | | | | |
| 3 | 1 | 4-arm-PEG-SG Ester 10K | 10,000 | 100 | 35 seconds | Phosphate | 37° C. | 20 |
| | 2 | TriLysine | 402 | 5.32 | | 0.1 Molar | | |
| | 3 | Phosphate Buffer pH 7.4, | | 0.5 ml | | pH 7.4 | | |
| | | 0.1 molar) | | | | | | |
| 4 | 1 | 4-arm-PEG-SAS 10K | 10,000 | 6.25 | 52 seconds | Phosphate | 37° C. | 28 |
| | 2 | 4-arm-PEG-SG Ester 10K | 10,000 | 18.75 | | 0.1 Molar | | |
| | 3 | 4-Arm-PEG-amine 10K | 10,000 | 25 | | pH 7.4 | | |
| | 4 | Phosphate Buffer pH 7.4, | | 0.5 ml | | | | |
| | | 0.1 molar) | | | | | | |
| 5 | 1 | 4-arm-PEG-SAS 10K | 10,000 | 25 | 30 seconds | Phosphate | 37° C. | 36 |
| | 2 | 4-arm-PEG-SG Ester 10K | 10,000 | 75 | | 0.1 Molar | | |
| | 3 | 4-Arm-PEG-amine 5K | 5,000 | 50 | | pH 7.4 | | |
| | 4 | Phosphate Buffer pH 7.4, | | 1.5 ml | | | | |
| | | 0.1 molar) | | | | | | |

TABLE 1B

| | | | | | | Gel Dissolution Time | | |
|---|---|---|---|---|---|---|---|---|
| | | | | | Gel Formation | Dissolution | Study | Dissolution |
| | | Materials | MW | Mass (mg) | Time | Buffer | Temperature | Time |
| 6 | 1 | 4-arm-PEG-SAS 10K | 10,000 | 25 | 50 seconds | Phosphate | 37º C. | 330 |
| | 2 | 4-arm-PEG-SG Ester 10K | 10,000 | 25 | | 0.1 Molar | | |
| | 3 | 4-Arm-PEG-amine 5K | 5,000 | 25 | | pH 7.4 | | |
| | 4 | Phosphate Buffer pH 7.4, | | 1.0 ml | | | | |
| | | 0.1 molar) | | | | | | |
| 7 | 1 | 4-arm-PEG-SAS 10K | 10,000 | 12.5 | 52 sec | Phosphate | 37° C. | 92 |
| | 2 | 4-arm-PEG-SG Ester 10K | 10,000 | 12.5 | | 0.1 Molar | | |
| | 3 | 4-Arm-PEG-amine 10K | 10,000 | 25 | | pH 7.4 | | |
| | 4 | Phosphate Buffer pH 7.4, | | 0.5 ml | | | | |
| | | 0.1 molar) | | | | | | |
| 8 | 1 | 4-arm-PEG-SAS 10K | 10,000 | 18.75 | 48 sec | Phosphate | 37° C. | 183 |
| | 2 | 4-arm-PEG-SG Ester 10K | 10,000 | 6.25 | | 0.1 Molar | | |
| | 3 | 4-Arm-PEG-amine 10K | 10,000 | 25 | | pH 7.4 | | |
| | 4 | Phosphate Buffer pH 7.4, | | 0.5 ml | | | | |
| | | 0.1 molar) | | | | | | |
| | | | | | | Gel Dissolution Time | | |
| | | | | | Gel Formation | Dissolution | Study | Dissolution |
| | | Materials | MW | Mass (mg) | Time | Buffer | Temperature | Time (days) |
| 9 | 1 | 4arm PEG-Thiol 10K | 10,000 | 25 | 75 sec | Phosphate | 37° C. | 18 |
| | 2 | 4-Arm PEG-Acrylate 20K | 20,000 | 50 | | 0.1 Molar | | |
| | 3 | Phosphate Buffer pH 7.73, | | 1.00 ml | | 7.4 pH | | |
| | | 0.1 molar) | | | | | | |
| 10 | 1 | 4arm PEG-SAS 10K | 10,000 | 25 | 78 seconds | Phosphate | 37° C. | 15 |
| | 2 | 4-arm-PEG-10K-ester amine | 10,000 | 25 | | 0.1 Molar | | |
| | 3 | Phosphate Buffer pH 7.73, | | 0.75 | | 7.4 pH | | |
| | | 0.1 molar) | | | | | | |

### Example 11: Formation of Gel Containing Liraglutide Molecule

Powder mixtures of 4arm PEG-SAS 10K (12.50 mg), 4-Arm PEG-SG Ester 10K (37.5 mg) and 4-arm-PEG-10K-amine (25 mg) were placed inside a 5 ml syringe. In another 5 ml syringe 15 mg of Liraglutide was dissolved in 1.5 ml sodium hydrogen phosphate buffer solution (0.1 molar, pH 7.4). The front ends of the two syringes were connected with a Luer lock connector and the contents were mixed by pressing the syringe plungers back and forth for about 10-20 seconds. The reactive mixture was pushed into one syringe. The Luer lock was disconnected and the contents were transferred into a 10 ml Falcon tube. The liquid formed the gel in about 75 seconds. The gel time was recorded as shown in the Table 2:

TABLE 2

| | | | | | Gel Dissolution Time | | |
|---|---|---|---|---|---|---|---|
| | | | | Gel Formation | Dissolution | Study | Dissolution |
| | Materials | MW | Mass (mg) | Time | Buffer | Temperature | Time |
| 1 | 4-arm-PEG-SAS 10K | 10,000 | 12.5 | 75 seconds | Phosphate | 37° C. | 30 |
| 2 | 4-arm-PEG-SG Ester 10K | 10,000 | 37.5 | | 0.1 Molar | | |
| 3 | 4-Arm-PEG-amine 10K | 10,000 | 50 | | pH 7.4 | | |
| 4 | Liraglutide powder | | 15 | | | | |
| 5 | Phosphate Buffer pH 7.4, | | 1.5 ml | | | | |
| | 0.1 molar) | | | | | | |

### Example 12: Elution Studies

The gel was dislodged from the bottom of the falcon tube and 2 ml buffer (pH 7.4, 0.1 molar) was added into the falcon tubes. The Falcon was placed in a water bath maintained at 37° C. After 24 hours, the liquid was drained from the falcon tube and replaced with another 2 ml of the buffer every day. The process was repeated till the sample dissolved completely in about 30 days. The collected samples were analyzed by a UV spectrometer after the calibration is performed and the elution amount determined.

Results: Almost no Liraglutide molecule was eluted as determined by UV spectrometer

### Repeat-1: Formation of Gel Containing GLP-1 Molecule

Powder mixtures of 4arm PEG-SAS 10K (12.50 mg), 4-Arm PEG-SG Ester 10K (37.5 mg) and 4-arm-PEG-10K-amine (25 mg) were placed inside a 5 ml syringe. In another 5 ml syringe 15 mg of Liraglutide was dissolved in 1.5 ml sodium hydrogen phosphate buffer solution (0.1 molar, pH 7.4). The front ends of the two syringes were connected with a Luer lock connector and the contents were mixed by pressing the syringe plungers back and forth for about 10-20 seconds. The reactive mixture was pushed into one syringe. The Luer lock was disconnected and the contents were transferred into a 10 ml Falcon tube. The liquid formed the gel in about 75 seconds. The gel time was recorded as shown in the Table 3:

TABLE 3

| | | | | | Gel Dissolution Time | | |
|---|---|---|---|---|---|---|---|
| | | | | Gel Formation | Dissolution | Study | Dissolution |
| | Materials | MW | Mass (mg) | Time | Buffer | Temperature | Time |
| 1 | 4-arm-PEG-SAS 10K | 10,000 | 12.5 | 78 seconds | Phosphate | 37° C. | 34 |
| 2 | 4-arm-PEG-SG Ester 10K | 10,000 | 37.5 | | 0.1 Molar | | |
| 3 | 4-Arm-PEG-amine 10K | 10,000 | 50 | | pH 7.4 | | |
| 4 | Liraglutide powder | | 15 | | | | |
| 5 | Phosphate Buffer pH 7.4, | | 1.5ml | | | | |
| | 0.1 molar) | | | | | | |

### Example 13: Elution Studies

The gel was dislodged from the bottom of the falcon tube and was cut into approximately 2 equal pieces. Both pieces were placed back into the falcon tube and 2 ml buffer (pH 7.4, 0.1 molar) was added into the falcon tubes. The Falcon tube was placed in a water bath maintained at 37° C. After 24 hours, the liquid was drained from the falcon tube and replaced with another 2 ml of the buffer every day. The process was repeated till the sample dissolved completely in about 30 days. The collected samples were analyzed by a UV spectrometer after the calibration is performed and the elution amount determined.

Result: GLP-1 samples were eluted for about 18 days but the amount of eluted concentration drops significantly after 18 days

### Repeat 2: Formation of Gel Containing GLP-1 Molecule

Powder mixtures of 4arm PEG-SAS 10K (12.50 mg), 4-Arm PEG-SG Ester 10K (37.5 mg) and 4-arm-PEG-10K-amine (25 mg) were placed inside a 5 ml syringe. In another 5 ml syringe 15 mg of Liraglutide was dissolved in 1.5 ml sodium hydrogen phosphate buffer solution (0.1 molar, pH 7.4). The front ends of the two syringes were connected with a Luer lock connector and the contents were mixed by pressing the syringe plungers back and forth for about 10-20 seconds. The reactive mixture was pushed into one syringe. The Luer lock was disconnected and the contents were transferred into a 10 ml Falcon tube. The liquid formed the gel in about 75 seconds. The gel time was recorded as shown in the Table 4:

TABLE 4

| | | | | | Gel Dissolution Time | | |
|---|---|---|---|---|---|---|---|
| | | | | Gel Formation | Dissolution | Study | Dissolution |
| | Materials | MW | Mass (mg) | Time | Buffer | Temperature | Time |
| 1 | 4-arm-PEG-SAS 10K | 10,000 | 12.5 | 72 seconds | Phosphate | 37° C. | 32 |
| 2 | 4-arm-PEG-SG Ester 10K | 10,000 | 37.5 | | 0.1 Molar | | |
| 3 | 4-Arm-PEG-amine 10K | 10,000 | 50 | | pH 7.4 | | |
| 4 | Liraglutide powder | | 15 | | | | |

| | | |
|---|---|---|
| 5 | Phosphate Buffer pH 7.4, | 1.5 ml |
| | 0.1 molar) | |

### Example 14: Elution Studies

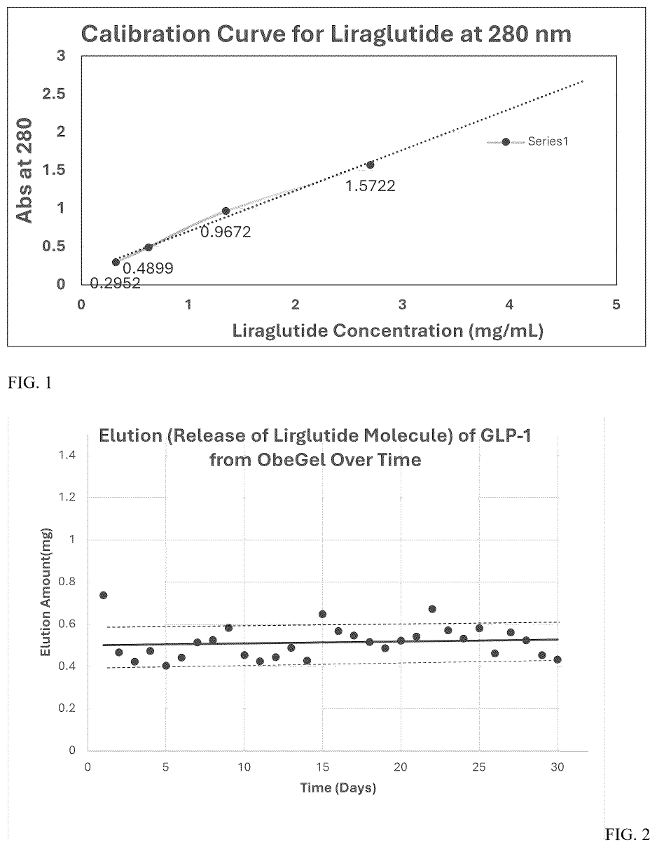

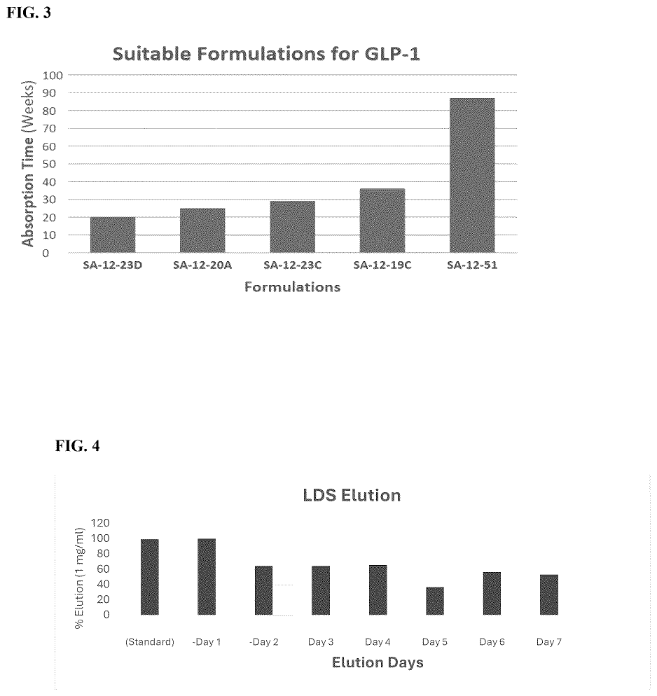

The gel was dislodged from the bottom of the falcon tube and was cut into approximately 4 equal pieces. All 4 pieces were placed back into the falcon tube and 2 ml buffer (pH 7.4, 0.1 molar) was added into the falcon tubes. To the sample formed in the Falcon tube, was added 2 ml buffer (0.1 molar, pH 7.4) and the Falcon tube was placed in a water bath maintained at 37° C. After 24 hours, the liquid was drained from the falcon tube and replaced with another 2 ml of the buffer every day. The process was repeated till the sample dissolved completely in about 30 days. The collected samples were analyzed by a UV spectrometer after the calibration is performed and the elution amount determined. The UV Spectrometer was calibrated. The calibration results are shown in FIGS. 1 and 2.

### Example 15: Clinical Trial for the Treatment of Obesity and Type 2 Diabetes to Evaluate the Efficacy and Safety of a Commercial Embodiment Named Slimsustain-X in Adults with Obesity and Type 2 Diabetes

The study goal is to evaluate the safety and efficacy of the pharmacological agent discussed above for the treatment of obesity and type 2 diabetes. The agent is designed to provide dual metabolic benefits through a unique mechanism of action, potentially offering improved weight management and glycemic control. The study aims to assess various dosing regimens to determine optimal therapeutic efficacy while minimizing side effects. The primary study endpoint is the mean percentage change in body weight from baseline to Week 24. Secondary endpoints include changes in HbA1c levels, fasting plasma glucose levels, proportion of participants achieving ≥5% and ≥10% weight loss, and changes in waist circumference and body fat percentage. Safety assessments include monitoring of adverse events, vital signs, ECG assessments, and laboratory tests for liver and kidney function. The 24-week randomized controlled trial (300 participants, BMI >30, HbA1c 7-9.5%) compares the agent against placebo with weight loss and glycemic control endpoints, following established regulatory frameworks for obesity drug development. The patient population for the study consists of 300 male and female adults (ages 18-65) diagnosed with obesity (BMI >30 kg/m\hat{ }2) and type 2 diabetes (HbA1c 7.0-9.5%). Inclusion criteria encompass adults aged 18-65 years with a BMI >30 kg/m², diagnosed with type 2 diabetes for at least 6 months, HbA1c between 7.0-9.5%, stable glucose-lowering therapy for at least 3 months, and willingness to follow dietary and exercise recommendations.

Prior to treatment, baseline assessments are conducted to determine disease activity and metabolic parameters. Participants are randomized in a 2:1 ratio to receive either Compound X (50 mg once daily) or a matched placebo. The study follows a double-blind design where participants, investigators, and data analysts are blinded to treatment assignment. Study parameters and adverse events are collected at baseline and at specified intervals throughout the 24-week trial period. Slimsustain-X trials should demonstrate weight loss exceeding 10% (current phase 2 benchmarks) with parallel HbA1c reductions ≥1%. Comparative data from SURMOUNT-1's 22.9% weight reduction at 176 weeks and Semaglutide's 12.1% in East Asian populations suggest high efficacy thresholds and demonstrate the dual benefits of Slimsustain-X in weight loss and glycemic control, addressing the needs for obesity and type 2 diabetes treatment.

1. A sustained drug delivery method for treating obesity and type 2 diabetes comprising:
preparing an injectable formulation by mixing a gel matrix comprising GRAS-approved polymers with a GLP-1 receptor agonist;
reacting 4-arm-PEG-SAS 10K, 4-arm-PEG-SG Ester 10K, 4-Arm-PEG-amine 10K in a phosphate liquid buffer for a percutaneous application to a mammalian subject;
forming an in-situ depot that releases the agonist over a predetermined period through controlled porosity of the gel matrix.

2. The method of claim 1 wherein the GRAS-approved polymers are selected from polyethylene glycol (PEG), poly(lactic-co-glycolic acid) (PLGA), poly lactide (PL), polyglycolide (PG), and hyaluronic acid (HA).

3. The method of claim 1 wherein pore formation in the gel matrix is triggered by light exposure or chemical activation.

4. The method of claim 1 wherein release duration is controlled by adjusting ratios of gel matrix components selected from:
4-arm-PEG-SAS 10K, 4-arm-PEG-SG Ester 10K, 4-Arm-PEG-amine 10K, and sodium hydrogen phosphate buffer (0.5-1.5 ml).

5. The method of claim 1 further comprising adjusting hydration levels between 10-95% w/v to control release kinetics.

6. The method of claim 1 further comprising combining the GLP-1 agonist with at least one additional weight loss agent selected from amylin analogs and leptin receptor agonists.

7. The method of claim 1 further comprising administering the formulation via subcutaneous injection in abdominal adipose tissue.

8. The method of claim 1 further comprising combining the GLP-1 agonist or other peptide molecules such semaglutide, tirzeptide, dulaglutide, extenide as that are similar in molecular weight that could be effective in other applications such as type 3c diabetes or pancreatic cancer etc.

9. The method of claim 1, comprising forming a biodegradable ester amine:

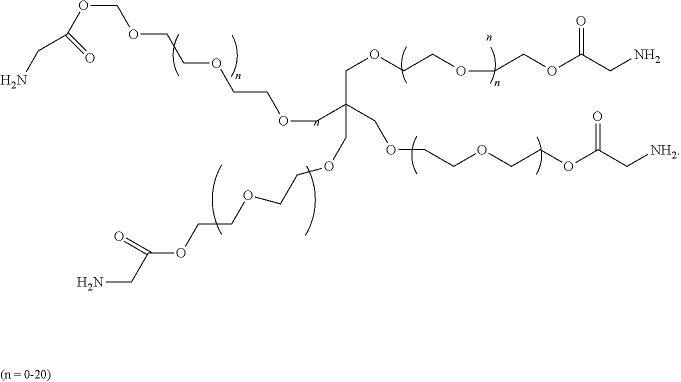

10. The method of claim 1, comprising triggering using light or chemicals to form:

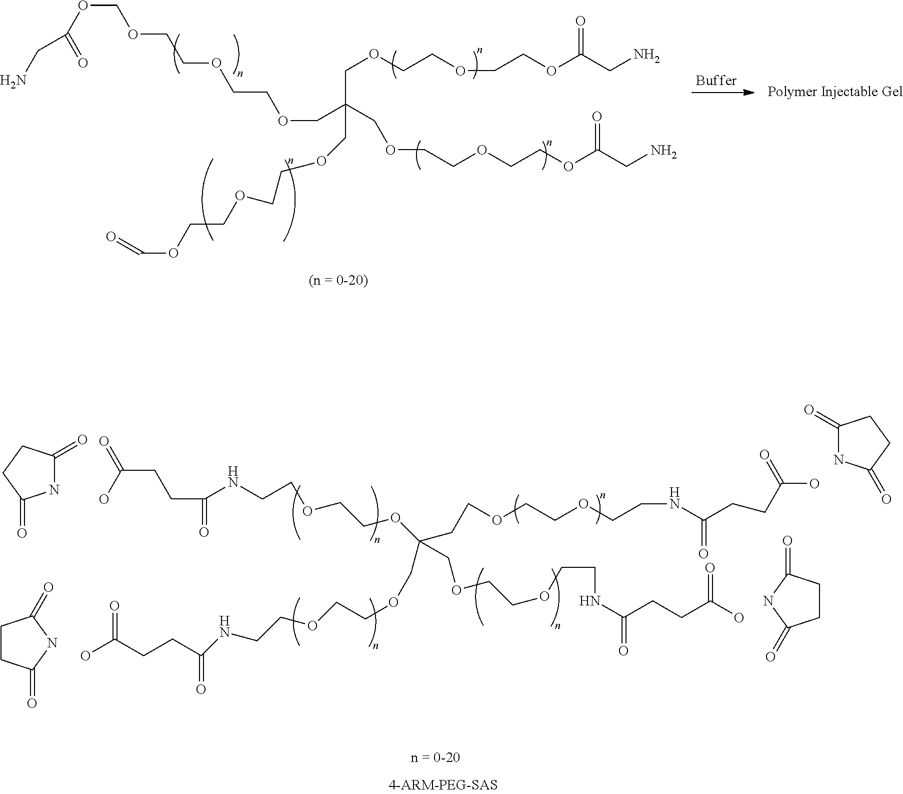

11. An injectable formulation, comprising a gel matrix from mixing a GRAS-approved polymers with a GLP-1 receptor agonist and reacting 4-Arm PEG-SG Ester with 4-arm-PEG-amine in a phosphate buffer for a percutaneous application to a mammalian subject, wherein the gel matrix releases the agonist over a predetermined period through controlled porosity of the gel matrix.

12. The formulation of claim 11, wherein the formulation comprises:

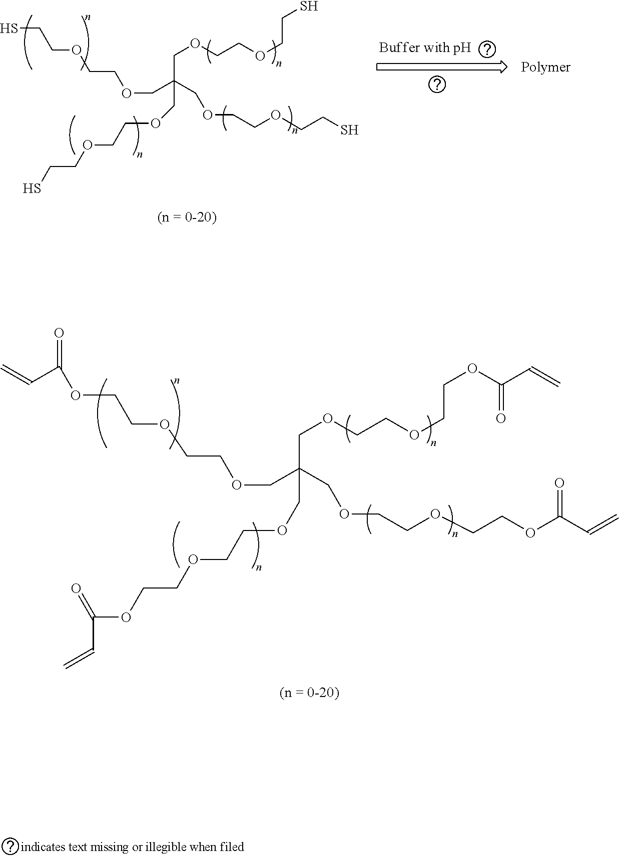

13. The formulation of claim 11, wherein the formulation comprises a biodegradable ester amine:

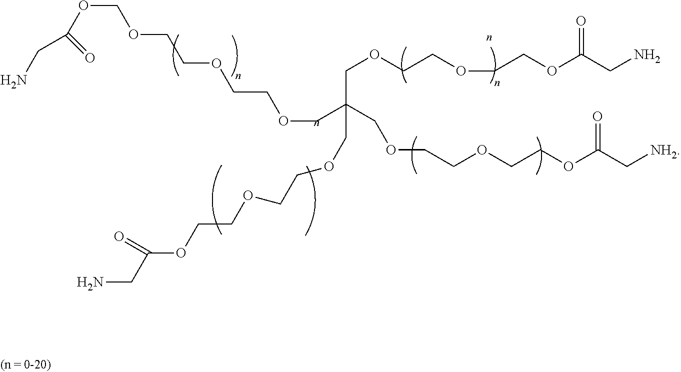

14. The formulation of claim 11, wherein a reaction is triggered by light or chemicals to form:

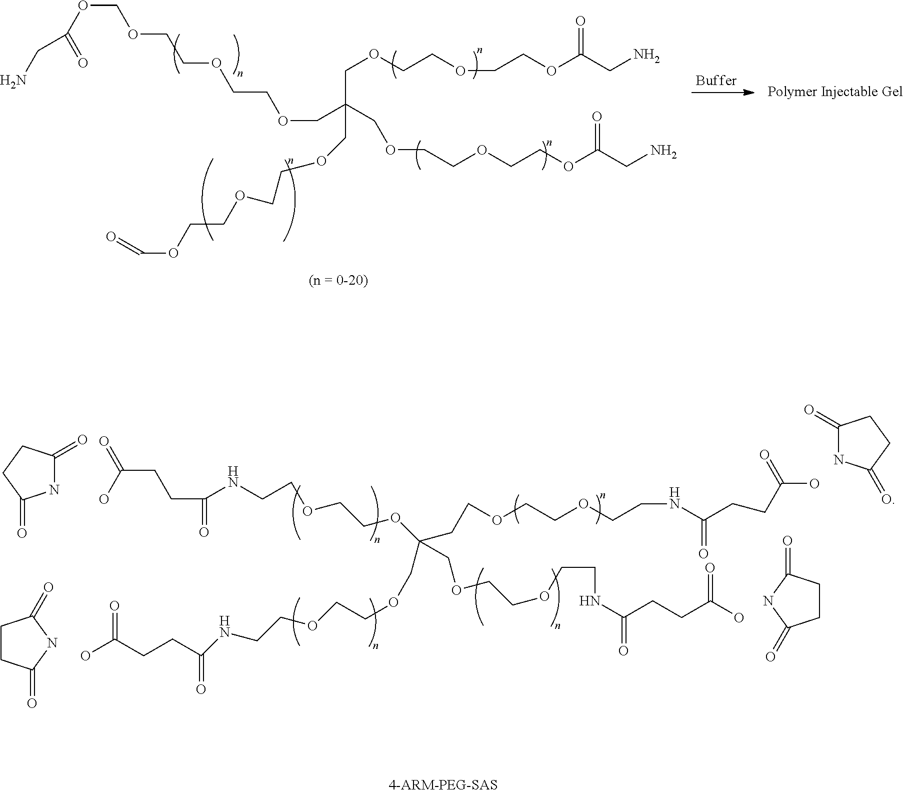

15. The formulation of claim 11, wherein the formulation comprises:

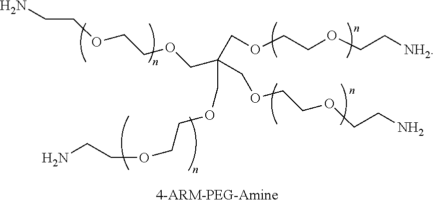

16. The formulation of claim 11, wherein the 4-Arm PEG-SG Ester with 4-arm-PEG-amine comprises 4-arm-PEG-SG Ester 10K+4-Arm-PEG-amine 5K or 4-arm-PEG-SG Ester 10K+TriLysine or 4-arm-PEG-SAS 10K+4-arm-PEG-SG Ester 10K+4-Arm-PEG-amine 10K or 4-arm-PEG-SAS 10K+4-arm-PEG-SG Ester 10K+4-Arm-PEG-amine 5K or 4arm PEG-Thiol 10K+4-Arm PEG-Acrylate 20K or 4arm PEG-SAS 10K+4-arm-PEG-10K-ester amine.

17. The formulation of claim 11, wherein the 4-Arm PEG-SG Ester with 4-arm-PEG-amine comprises 4-arm-PEG-SG Ester 10K+TriLysine or 4arm PEG-Thiol 10K+4-Arm PEG-Acrylate 20K or 4arm PEG-SAS 10K+4-arm-PEG-10K-ester amine.
